(19) United States
(12) Patent Application Publication
(10) Pub. No.: US 2016/0270765 A1
(43) Pub. Date: Sep. 22, 2016

(54) ULTRASONIC DIAGNOSTIC APPARATUS AND BIOMETRIC EXAMINATION APPARATUS

(71) Applicant: KABUSHIKI KAISHA TOSHIBA, Minato-ku (JP)

(72) Inventors: Satoshi TAKAYAMA, Kawasaki (JP); Taeko URANO, Kawasaki (JP); Tsutomu NAKANISHI, Yokohama (JP); Kenji NAKAMURA, Kawasaki (JP)

(73) Assignee: KABUSHIKI KAISHA TOSHIBA, Minato-ku (JP)

(21) Appl. No.: 15/067,994

(22) Filed: Mar. 11, 2016

(30) Foreign Application Priority Data
Mar. 17, 2015 (JP) 2015-053522

(51) Int. Cl. A61B 8/08; A61B 5/00; A61B 8/00

(52) U.S. Cl. CPC A61B 8/5207; A61B 8/463; A61B 5/0059; A61B 5/441

(57) ABSTRACT

According to one embodiment, an ultrasonic diagnostic apparatus comprises an ultrasonic probe, image generation circuitry, an optical probe, at least one integrated solid-state image sensor and analysis circuitry. The image generation circuitry generates an ultrasonic image by using the ultrasonic wave. The an optical probe is integrally provided with the ultrasonic probe and including at least one light emission circuitry to apply light in an absorption wavelength band of a biological component from a surrounding of the ultrasonic transmission/reception surface onto the inside of the object and a plurality of optical detection circuits to detect an intensity of light having a specific wavelength which is applied from the at least one light emission circuitry and diffused/reflected by the inside of the object. The analysis circuitry is configured to analyze a change in the light intensity detected by the each optical detection circuit.

### CROSS-REFERENCE TO RELATED APPLICATIONS

This application is based upon and claims the benefit of priority from the prior Japanese Patent Application No. 2015-053522, filed Mar. 17, 2015 the entire contents which are incorporated herein by reference.

### FIELD

Embodiments described herein relate generally to an ultrasonic diagnostic apparatus and a biometric examination apparatus.

### BACKGROUND

An embodiment of the present invention relates to an ultrasonic diagnostic apparatus and a biometric examination apparatus.

There are various techniques of noninvasively measuring the inside of the living body. Optical measurement, as one of such techniques, has merits of being free from the problem of exposure to radiation and capable of selecting a compound as a measurement target by selecting a wavelength. A general biometrical measurement apparatus is designed to emit the inside of the living body with light while a light emission unit is pressed against the surface of the skin of the living body and to measure transmitted or reflected light which is transmitted through the skin again and emerges out of the living body and calculate various types of biometrical information based on the measurements. The presence of an abnormal tissue in the living body is determined by optical measurement based on the difference in light absorption coefficient between the abnormal tissue and a normal tissue. That is, the difference in absorption coefficient between normal and abnormal tissues in the living body leads to a difference in the amount of light detected based on a difference in the amount of light absorbed. Therefore, solving an inverse problem from the amount of light detected can obtain the absorption coefficient of an abnormal tissue. A characteristic of the abnormal tissue can be discriminated from the obtained absorption coefficient. In addition, the measurement position and depth are analyzed from the measured light. Such analysis techniques include a technique (spatial decomposition method) of adjusting the distance between a light emission unit (or light source) and a detector, a technique (time decomposition method) of obtaining depth information from a difference in the arrival time of light by using a light source whose intensity changes with time, and a method as a combination of them. These analysis methods implement a biometrical measurement apparatus which can acquire high quality signals. However, the technique of imaging information based on light in the living body has the problem of a low spatial resolution. In addition, in order to obtain correct position information from the result of detecting reflected light, it is necessary to compute many data using a complicated algorithm. This makes it impossible to perform real time determination.

Highly feasible applications of optical biometrical measurement include breast cancer examination. As described above, however, since performing optical measurement alone leads to problems in terms of resolution and analysis time, it is preferable to improve examination performance by using this technique in combination of another modality. For this reason, the present inventors have proposed a scheme of compensating for the low spatial resolution of light by using the morphological information of ultrasonic echoes. Although this scheme is expected to be capable of discriminating morphological characteristics in living body tissues and the partial distribution of morphological characteristic portions within a shorter time than in a related art, there is still room for improvement in immediate determination.

Breast cancer is one of the causes of women death. Breast cancer screening and early diagnosis have very high values in terms of reducing mortality rate and suppressing the cost of health care.

Existing methods include palpation of breast tissues and X ray imaging for searching for suspected tissue deformation. If there is a suspected portion in an X ray photograph, ultrasonic imaging is performed, and surgical tissue examination is further performed. A series of these examinations require much time to reach a final conclusion. In addition, since premenopausal young women have many mammary glands, high sensitivity is difficult to obtain in X ray imaging. Therefore, screening using ultrasonic imaging has great significance for the young generation, in particular.

In general, in ultrasonic imaging, a certified operator acquires ultrasonic still images, and an expert interpreter (a plurality of interpreters in some cases) makes determination from morphological information on the images. When performing medical examination, the maximum number of persons subjected to screening per operator per day is 50 in consideration of the risk of oversights caused by the fatigue and lack of concentration of the operator.

In order to acquire a still image capturing a morphological characteristic in ultrasonic imaging, it is very important for the operator to have knowledge and experience. High skill is also required to perform accurate and quick screening. For example, standard examination times per object are 5 to 10 min. However, it sometimes takes more time for screening depending on the skill of an operator. That is, in screening based on current ultrasonic imaging, the accuracy of image acquisition may vary depending on the levels of skill of operators. When acquiring images, the operator needs to keep paying close attention to images. Besides, he/she takes charge of making determination by himself/herself, and hence a heavy metal strain is imposed on him/her even if he/she is a skilled operator. Although there is available a scheme of acquiring all image information from a moving image, there is no established technique for mechanical search using image recognition. For this reason, an interpreter searches a moving image for still images. In this case, a heavy burden is imposed on the interpreter.

In order to solve the above problem, the present applicant has proposed an apparatus with the concept of complement of ultrasonic echo diagnosis by using a compact optical examination system designed to reduce a burden on a technician by guiding the measurement position of an ultrasonic echo probe in a plane direction based on the metabolic information of the living body which is obtained by optical measurement. FIG. 18 shows the light absorption spectra of oxygenated hemoglobin and deoxygenated hemoglobin. In general, deoxygenated hemoglobin in a malignant tumor region is higher in ratio than in a healthy region, and hence an analysis result on the absorption of deoxygenated hemoglobin is one of the bases for determining the degree malignancy of a target region in optical biometrical examination. The wavelength regions of light suitable for the light absorption measurement of deoxygenated hemoglobin are 740 nm to 790 nm in the near infrared light region and 650 nm to 690 nm in the red light region. The wavelength region of light suitable for the light absorption measurement of oxygenated hemoglobin is 830 nm to 900 nm in the near infrared light region. The wavelength region of light to identify a total hemoglobin amount is, for example, 800 nm to 820 nm in the near infrared light region. Specific light sources include an LED and an LD. In consideration of absorption wavelength of other biological components such as water, fat, and melanin and biodistributions, an output light intensity and a half width must be properly selected for a light source.

One of the problems in conventionally proposed biometrical measurement apparatuses is that a measurement target to which an ultrasonic echo probe is guided by an optical measurement system is not always located immediately below the probe. This problem originates from the selection of an arrangement in which the longitudinal symmetrical axis of the ultrasonic echo piezoelectric probe is orthogonal to a symmetrical axis of the optical measurement system.

In order to solve this problem, the present inventors have proposed the arrangement of a probe configured such that a symmetrical axis of an optical measurement system substantially coincides with the longitudinal axis of an ultrasonic echo piezoelectric probe (a symmetrical axis of an optical measurement system is arranged to be parallel to the longitudinal direction of the piezoelectric probe and fall within its short side). In such a typical probe arrangement, a light introducing unit is arranged at a position near a short side of the piezoelectric probe, and a plurality of detection units are symmetrically arranged near a probe short side. This avoids the problem that a measurement target is positioned outside the piezoelectric probe.

In this arrangement, however, since a measurement target is positioned at an end of an echo image, it is not possible to meet the need to guide a target to the central position to perform examination while rotating the probe. In order to solve this problem, the present inventors have selected an arrangement in which a plurality of light sources are arranged near the central position of the piezoelectric probe so as to be symmetrical with respect to an axis in the long side direction, and a plurality of detection units to be paired with the light sources are arranged symmetrically with respect to the axis in the short side direction of the piezoelectric probe. This arrangement makes it possible to guide a measurement target to the central position of an echo probe. In addition, the present inventors have also proposed an optical detection system which has been improved in terms of the implementation of a multiple light source scheme in order to solve the problem that the amount of light absorbed changes because the probe is brought into pressure contact with the skin.

This probe arrangement is suited to guide an abnormal region to a position immediately below the middle of the probe, but can only roughly estimate the depth and size of a region. For this reason, when, for example, it is necessary to perform image examination with a spatial resolution of 5 mm, data obtained by one measurement is not enough for the examination. Obviously, the problem can be solved by greatly increasing the numbers of light incident positions and detection positions. This requires an enormous calculation amount and an enormous calculation time. In addition, as the number of channels increases accordingly, the detection system increases in size. This makes it impossible to meet the need for a compact system which complements ultrasonic echoes.

To solve this problem, imaging can be implemented by acquiring the necessary number of data by performing a plurality of measurements with an arrangement whose relative position is properly changed. However, it is highly difficult to perform accurate positioning in biometrical examination. In addition, when the surface of the living body is scanned with a probe, the distribution of blood changes by the pressure on the skin. For this reason, bringing the probe into contact with the skin with a high repeatability also increases the difficulty in measurement. As described above, examination technicians are required to have high skills and techniques.

In this arrangement, however, since a measurement target is positioned at an end of an echo image, it is not possible to meet the need to guide a target to the central position to perform examination while rotating the probe. In order to solve this problem, the present inventors have selected an arrangement in which a plurality of light sources are arranged near the central position of the piezoelectric probe so as to be symmetrical with respect to an axis in the long side direction, and a plurality of detection units to be paired with the light sources are arranged symmetrically with respect to the axis in the short side direction of the piezoelectric probe. This arrangement makes it possible to guide a measurement target to the central position of an echo probe. In addition, the present inventors have also proposed an optical detection system which has been improved in terms of the implementation of a multiple light source scheme in order to solve the problem that the amount of light absorbed changes because the probe is brought into pressure contact with the skin.

This probe arrangement is suited to guide an abnormal region to a position immediately below the middle of the probe, but can only roughly estimate the depth and size of a region. For this reason, when, for example, it is necessary to perform image examination with a spatial resolution of 5 mm, data obtained by one measurement is not enough for the examination. Obviously, the problem can be solved by greatly increasing the numbers of light incident positions and detection positions. This requires an enormous calculation amount and an enormous calculation time. In addition, as the number of channels increases accordingly, the detection system increases in size. This makes it impossible to meet the need for a compact system which complements ultrasonic echoes.

To solve this problem, imaging can be implemented by acquiring the necessary number of data by performing a plurality of measurements with an arrangement whose relative position is properly changed. However, it is highly difficult to perform accurate positioning in biometrical examination. In addition, when the surface of the living body is scanned with a probe, the distribution of blood changes by the pressure on the skin. For this reason, bringing the probe into contact with the skin with a high repeatability also increases the difficulty in measurement. As described above, examination technicians are required to have high skills and techniques.

### BRIEF DESCRIPTION OF THE DRAWING

FIG. 1 is a block diagram showing the arrangement of a biometric examination apparatus 1 according to an embodiment;

FIG. 2 is a block diagram showing the arrangement of a biometric measurement apparatus 4 including an optical probe 40 and an optical measurement processing unit 42;

FIG. 3 is a view when a probe P is seen from the object contact surface side, showing an example of the placement of light emission units 400, optical detection units 401, and an integrated solid-state image sensor 403a with respect to the ultrasonic transmission/reception surface of an ultrasonic probe 12;

FIG. 4 is a view showing an example of the arrangement of the optical measurement system of a biometric measurement apparatus 4;

FIG. 5 is a view showing an example of the arrangement of the optical measurement system of a biometric measurement apparatus 4;

FIGS. 6A and 6B are views for explaining measurement processing by the biometric measurement apparatus 4;

FIG. 7 is a view showing an example of display of optical measurement results and support information for guiding the probe P in the ultrasonic diagnostic apparatus 1 according to this embodiment;

FIG. 8 is a view showing another example of display of optical measurement results and support information for guiding the probe P in the ultrasonic diagnostic apparatus 1 according to this embodiment;

FIG. 9 is a block diagram showing the arrangement of the biometric measurement apparatus 4 according to the first modification;

FIG. 10 is a view showing an example of the arrangement of the optical measurement system of the biometric measurement apparatus 4 shown in FIG. 9;

FIG. 11 is a view when the probe P according to the second modification is seen from the object contact surface side;

FIGS. 12A and 12B are views when the probe P according to the third modification is seen from the object contact surface side;

FIG. 13 is a view showing another example of the arrangement of the optical measurement system of the biometric measurement apparatus 4; and

FIG. 14 is a graph showing the light absorption spectra of oxygenated hemoglobin and deoxygenated hemoglobin.

### DETAILED DESCRIPTION

According to one embodiment, an ultrasonic diagnostic apparatus comprises an ultrasonic probe, image generation circuitry, an optical probe, at least one integrated solid-state image sensor and analysis circuitry. The ultrasonic probe transmits an ultrasonic wave from an ultrasonic transmission/reception surface to an object and receive an ultrasonic wave reflected by an inside of the object via the ultrasonic transmission/reception surface. The image generation circuitry generates an ultrasonic image by using the ultrasonic wave received by the ultrasonic probe. The an optical probe is integrally provided with the ultrasonic probe and including at least one light emission circuitry to apply light in an absorption wavelength band of a biological component from a surrounding of the ultrasonic transmission/reception surface onto the inside of the object, and a plurality of optical detection circuits to detect an intensity of light having a specific wavelength which is applied from the at least one light emission circuitry and diffused/reflected by the inside of the object. The at least one integrated solid-state image sensor and analysis circuitry obtains data with respect to intradermal information of the object. The analysis circuitry analyzes a change in the light intensity detected by the each optical detection circuit.

An embodiment will be described below with reference to the accompanying drawing. Note that the same reference numerals in the following description denote constituent elements having almost the same functions and arrangements, and a repetitive description will be made only when required.

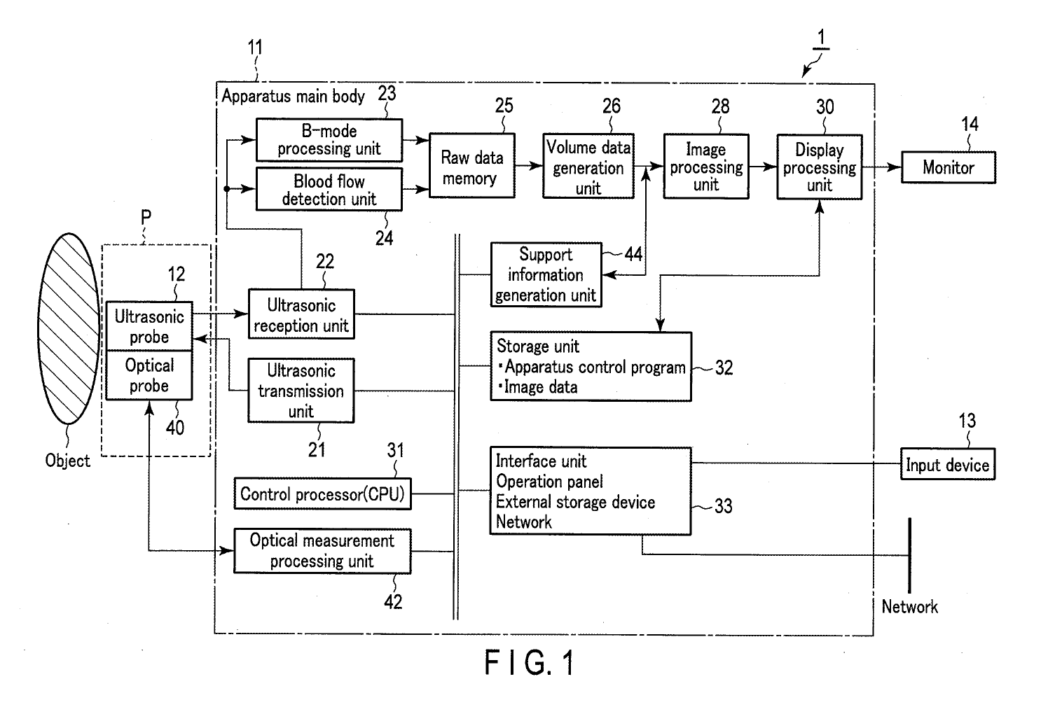

FIG. 1 is a block diagram showing the arrangement of an ultrasonic diagnostic apparatus 1 according to this embodiment. The ultrasonic diagnostic apparatus 1 shown in FIG. 1 includes an ultrasonic probe 12, an input device 13, a monitor 14, an ultrasonic transmission unit 21, an ultrasonic reception unit 22, a B-mode processing unit 23, a blood flow detection unit 24, a raw data memory 25, a volume data generation unit 26, an image processing unit 28, a display processing unit 30, a control processor (CPU) 31, a storage unit 32, and an interface unit 33. The ultrasonic diagnostic apparatus 1 according to the embodiment further includes an optical probe 40 and an optical measurement processing unit 42, which serve to implement a biometric measurement apparatus 4, and a scanning support information creation unit 44 which generates support information for supporting the placement operation of the ultrasonic probe 12.

Note that this embodiment will exemplify the ultrasonic diagnostic apparatus 1 incorporating the biometric measurement apparatus (integrated with the biometric measurement apparatus). However, this is not exhaustive, and the biometric measurement apparatus and the ultrasonic diagnostic apparatus may be separate structures.

The ultrasonic probe 12 is a device (probe) which transmits ultrasonic waves to an object, typically the living body, and receives reflected waves from the object based on the transmitted ultrasonic waves. The ultrasonic probe 12 has, on its distal end, an array of a plurality of piezoelectric transducers, a matching layer, a backing member, and the like. The piezoelectric transducers transmit ultrasonic waves in a desired direction in a scan region based on driving signals from the ultrasonic transmission unit 21, and convert reflected waves from the object into electrical signals. The matching layer is an intermediate layer which is provided for the piezoelectric transducers to make ultrasonic energy efficiently propagate. The backing member prevents ultrasonic waves from propagating backward from the piezoelectric transducers. When the ultrasonic probe 12 transmits an ultrasonic wave to the object, the transmitted ultrasonic wave is sequentially reflected by a discontinuity surface of acoustic impedance of internal body tissue, and is received as an echo signal by the ultrasonic probe 12. The amplitude of this echo signal depends on an acoustic impedance difference on the discontinuity surface by which the echo signal is reflected. The echo produced when a transmitted ultrasonic pulse is reflected by a moving blood flow is subjected to a frequency shift depending on the velocity component of the moving body in the ultrasonic transmission/reception direction by the Doppler effect.

Note that in this embodiment, the ultrasonic probe 12 is a one-dimensional array probe having a plurality of ultrasonic transducers arrayed along a predetermined direction. However, this is not exhaustive, and the ultrasonic probe 12 may be a two-dimensional array probe (i.e., a probe having ultrasonic transducers arranged in the form of a two-dimensional matrix) or a mechanical 4D probe (i.e., a probe which can execute ultrasonic scanning while mechanically swinging an ultrasonic transducer array), which can acquire volume data.

The input device 13 is connected to an apparatus body 11 and includes various types of switches which are used to input, to the apparatus body 11, various types of instructions, conditions, an instruction to set a region of interest (ROI), various types of image quality condition setting instructions, and the like from an operator, a switch for switching between a rough search mode and a fine adjustment mode which will be described later, buttons, a trackball, a mouse, and a keyboard. In addition, the input device 13 includes a button or the like for instructing the timing to capture paracentesis information including the position of the tip of a puncture needle in a paracentesis support function.

The monitor 14 displays morphological information and blood flow information in the living body as images based on video signals from the display processing unit 30.

The ultrasonic transmission unit 21 is circuitry which includes trigger generation circuitry, delay circuitry, and pulser circuitry (none of which are shown). The trigger generation circuitry repetitively generates trigger pulses for the formation of transmission ultrasonic waves at a predetermined rate frequency fr Hz (period: 1/fr sec). The delay circuitry gives each trigger pulse a delay time necessary to focus an ultrasonic wave into a beam and determine transmission directivity for each channel. The pulse circuitry applies a driving pulse to the probe 12 at the timing based on this trigger pulse.

The ultrasonic reception unit 22 is a circuitry which includes amplifier circuitry, A/D converter, delay circuitry, and adder (none of which are shown). The amplifier circuitry amplifies an echo signal received via the probe 12 for each channel. The A/D converter converts each analog echo signal into a digital echo signal. The delay circuitry gives the digitally converted echo signals delay times necessary to determine reception directivities and perform reception dynamic focusing. The adder then performs addition processing for the signals. With this addition, a reflection component from a direction corresponding to the reception directivity of the echo signal is enhanced to form a composite beam for ultrasonic transmission/reception in accordance with reception directivity and transmission directivity.

The B-mode processing unit 23 is circuitry which receives an echo signal from the reception unit 22, and performs logarithmic amplification, envelope detection processing, and the like for the signal to generate data whose signal intensity is expressed by a luminance level.

The blood flow detection unit 24 is circuitry which extracts a blood flow signal from the echo signal received from the ultrasonic reception unit 22, and generates blood flow data. In general, the blood flow detection unit 24 extracts a blood flow by CFM (Color Flow Mapping). In this case, the blood flow detection unit 24 analyzes the blood flow signal to obtain blood flow information such as mean velocities, variances, and powers as blood flow data at multiple points.

The raw data memory 25 generates B-mode raw data as B-mode data on three-dimensional ultrasonic scanning lines by using a plurality of B-mode data received from the B-mode processing unit 23. The raw data memory 25 generates blood flow raw data as blood flow data on three-dimensional ultrasonic scanning lines by using a plurality of blood flow data received from the blood flow detection unit 24. Note that for the purpose of reducing noise or smooth concatenation of images, a three-dimensional filter may be inserted after the raw data memory 25 to perform spatial smoothing.

The volume data generation unit 26 is circuitry which generates B-mode volume data or blood flow volume data by executing raw/voxel conversion including interpolation processing in consideration of spatial positional information.

The image processing unit 28 is circuitry which performs predetermined image processing such as volume rendering, MPR (Multi Planar Reconstruction), and MIP (Maximum Intensity Projection) for the volume data received from the volume data generation unit 26.

Note that for the purpose of reducing noise or smooth concatenation of images, a two-dimensional filter may be inserted after the image processing unit 28 to perform spatial smoothing.

The display processing unit 30 is processing circuitry which executes various types of processing associated with a dynamic range, luminance (brightness), contrast, γ curve correction, RGB conversion, and the like for various types of image data generated/processed by the image processing unit 28.

The control processor (CPU) 31 has the function of an information processing apparatus (computer) and controls the operation of each constituent element. The control processor 31 also executes processing in accordance with an ultrasonic probe operation support function (to be described later).

The storage unit 32 includes, for example, a semiconductor storage device such as a Flash SSD (Solid State Disk) serving as a semiconductor storage element, and an HDD (Hard Disk Drive). The storage unit 32 stores a dedicated program for implementing the ultrasonic probe operation support function (to be described later), obtained volume data, diagnosis information (patient ID, findings by doctors, and the like), a diagnostic protocol, transmission/reception conditions, and other data groups. The storage unit 32 is also used to store images in the image memory (not shown), as needed. It is possible to transfer data in the storage unit 32 to an external peripheral device via the interface unit 33. In addition, the storage unit 32 stores information concerning the moving distance of the probe P (to be described later), measurement data obtained by optical measurement, ultrasonic probe operation support information, and ultrasonic image data in association with each other for each measurement position.

The interface unit 33 is an interface circuitry which is associated with the input device 13, a network, and a new external storage device (not shown). In addition, an external biometric measurement apparatus can be connected to this ultrasonic diagnostic apparatus main body 11 via the interface unit 33. The interface unit 33 can transfer data such as ultrasonic images obtained by this apparatus, analysis results, and the like to other apparatuses via a network.

The scanning support information creation unit 44 is circuitry which calculates at least one the degree of adhesion between the ultrasonic probe 12 and the surface of an object and the three-dimensional azimuth and distance (proximity) of an abnormal region in an object based on the three-dimensional position and distance of the abnormal region which are acquired at each measurement position upon movement of the probe P. The scanning support information creation unit 44 then supports the operation of the ultrasonic probe by generating and outputting support information for more favorably inducing the position, orientation, posture, pressure, and the like of the ultrasonic probe 12 with respect to the object and the diagnostic target region based on the calculation result, thereby supporting the scanning operation of the ultrasonic probe. Note that as specific processing performed by the scanning support information creation unit 44, for example, the technique disclosed in Jpn. Pat. Appln. KOKAI Publication No. 2014-110878, the entire contents of which are incorporated herein by reference, can be adopted. The monitor 14 displays the created scanning support information in a predetermined form.

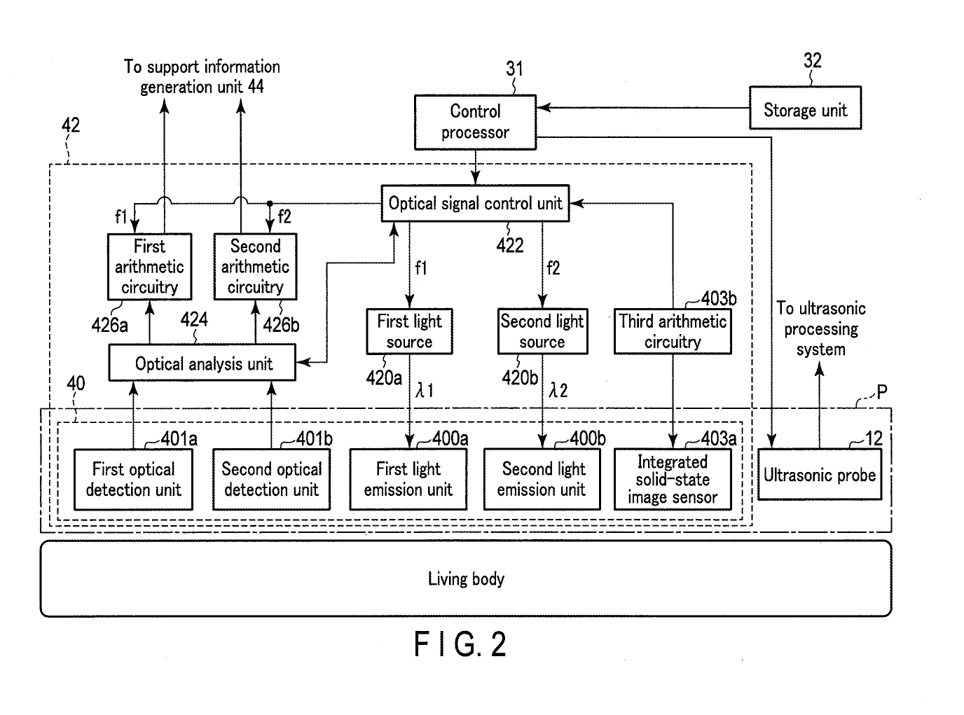

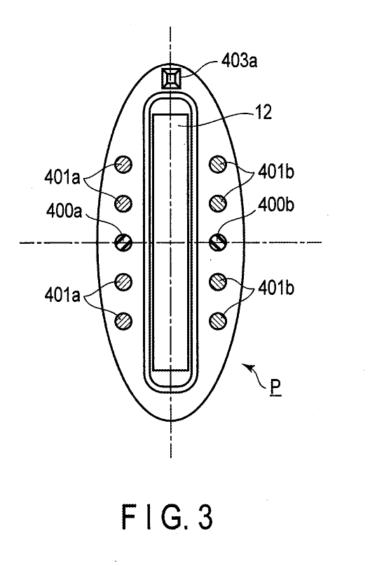

FIG. 2 is a block diagram of the biometric measurement apparatus 4 including the optical probe 40 and the optical measurement processing unit 42. FIG. 3 is a view when the probe P is seen from the object contact surface side, showing an example of the placement of light emission units 400, optical detection units 401, and an integrated solid-state image sensor 403a with respect to the ultrasonic transmission/reception surface of an ultrasonic probe 12.

As shown in FIGS. 2 and 3, the optical probe 40 includes at least one light emission unit 400 (a first light emission unit 400a and a second light emission unit 400b arranged on the two sides of the middle of the ultrasonic transmission/reception surface of the ultrasonic probe 12 in the case shown in FIG. 3) which are circuits, and the plurality of optical detection units 401 which are circuits and arranged so as to sandwich the first light emission units 400a and the second light emission unit 400b on the two sides of the middle of the ultrasonic transmission/reception surface of the ultrasonic probe 12. The plurality of light emission units 400 and the plurality of optical detection units 401 are line-symmetrically arranged with respect to the central axis of the ultrasonic transmission/reception surface of the ultrasonic probe 12 in the longitudinal direction and arranged at equal intervals along the longitudinal direction of the ultrasonic transmission/reception surface. In addition, the integrated solid-state image sensor 403a is arranged near a short side of the ultrasonic transmission/reception surface. This is merely an example. For example, there are modifications in which the positions and number of the integrated solid-state image sensors 403a are changed and in which the positional intervals between the above units are adjusted in consideration of the distances to the optical detection units 401 which they face.

The first light emission unit 400a and the second light emission unit 400b respectively emit an object with light (near-infrared light) generated by a first light source 420a and a second light source 420b. Different driving frequencies f1 and f2 are respectively supplied to the first light emission unit 400a and the second light emission unit 400b. As a result, the first light emission unit 400a and the second light emission unit 400b respectively emit the object with light beams having specific wavelengths at the different driving frequencies.

The first light source 420a and the second light source 420b are light-emitting elements such as semiconductor lasers, light-emitting diodes, solid-state lasers, or gas lasers which generate light having a wavelength exhibiting low biological absorption (e.g., light in the wavelength range of 600 nm to 1,800 nm, which is near the wavelength band called the biological window) and light having a specific wavelength exhibiting an increase in the amount of absorption of light in an abnormal region (e.g., light in the wavelength range of 750 nm to 850 nm, which falls within the wavelength band called the biological window and at which hemoglobin in blood absorbs light). Light beams generated by the first light source 420a and the second light source 420b are respectively supplied to the first light emission unit 400a and the second light emission unit 400b via, for example, an optical waveguide formed from an optical fiber or thin-film optical waveguide. Different driving frequencies are supplied to the first light emission unit 400a and the second light emission unit 400b. As a result, the first light emission unit 400a and the second light emission unit 400b emit the object with light beams having specific wavelengths at the different driving frequencies. Assume that in this embodiment, the first light source 420a and the second light source 420b generate light beams having the same wavelength. However, this is not exhaustive, and the first light source 420a and the second light source 420b may generate light beams having different wavelengths, as described later.

The plurality of optical detection units 401 are circuits and formed from a plurality of detection elements which have detection surfaces formed from, for example, end portions of optical fibers, and photoelectrically convert reflected light beams from the inside of the object which are input from the detection surfaces via optical waveguide portions. As each detection element, for example, a CCD, APD, photomultiplier tube, or the like can be adopted, as well as a light-receiving element such as a photodiode or phototransistor. The contact surfaces of each light emission unit 400 and each optical detection unit 401 with respect to the object may be provided with optical matching layers.

The integrated solid-state image sensor 403a is an image sensor formed from a CCD, CMOS, or the like, and executes imaging based on incident light. Image data obtained by imaging is sequentially output to third arithmetic circuitry 403b. In this case, data obtained by the integrated solid-state image sensor 403a is intradermal information of the object (corresponding to the amount of light absorbed depending on an intradermal composition distribution). The reason why intradermal information of the object is set as an imaging target is that information on the surface of the skin becomes uncertain because the surface of the object is coated with ultrasonic gel in ultrasonic image diagnosis.

Note that in the case shown in FIG. 3, external light (environmental light) is a light source for the integrated solid-state image sensor 403a. For this reason, the probe P includes no light source dedicated to imaging by the integrated solid-state image sensor 403a. This arrangement has the merit of being capable of simplifying the arrangement of the probe P, but has the demerit of adding large noise to a signal obtained by the integrated solid-state image sensor 403a.

The optical signal control unit 422 is a circuitry and dynamically or statically controls the biometric measurement apparatus 4. For example, the optical signal control unit 422 controls the first light source 420a and the second light source 420b (the first light source 420a and the second light source 420b have different driving frequencies in this embodiment, in particular) under the control of the control processor 31 of the ultrasonic diagnostic apparatus 1 so as to make the first light emission unit 400a and the second light emission unit 400b emit the object with light at predetermined timings, predetermined frequencies, intensities, and an intensity variation period T. In addition, the optical signal control unit 422 outputs a moving distance and the like (i.e. measurement position information) from a reference position to an optical analysis unit 424 at the timings of the execution of optical measurements (for example, the driving timings of the first light source 420a and the second light source 420b). Furthermore, the optical signal control unit 422 controls an optical analysis unit 424 so as to execute analysis processing corresponding to light with a predetermined driving period at a predetermined timing.

The optical analysis unit 424 is circuitry which includes a multi-channel lock-in amplifier. The optical analysis unit 424 selects period f1 and f2, detects only predetermined signals, and amplifies them. The optical analysis unit 424 then converts the signals into digital signals. The optical analysis unit 424 also analyzes a change in the intensity of detected light between the optical detection units 401. This analysis is executed for each measurement data acquired at each measurement position.

Based on a change in the intensity of detected light between the optical detection units 401, which is obtained at each measurement position, first arithmetic circuitry 426a calculates the three-dimensional position and distance of an abnormal region indicating a predetermined light absorption coefficient in the object (e.g., a region which absorbs light with a specific wavelength more than a normal tissue) with reference to the degrees of adhesion between the plurality of optical detection units 401 and the surface of the object, the depth of the abnormal region from the surface of the object, and a predetermined position (e.g., the light emission unit 400, the ultrasonic transmission/reception surface center of the ultrasonic probe 12, or the like). The calculation results obtained by the first arithmetic circuitry 426a are sent to the scanning support information creation unit 44 for each measurement position. Note that the arrangement and function of second arithmetic circumferential 426b are substantially the same as those of the first arithmetic circuitry 426a.

The third arithmetic circuitry 403b sequentially calculates the moving distance, coordinates, and moving direction of the probe P with reference to, for example, an initial position by using image data sequentially output from the integrated solid-state image sensor 403a. The calculated moving amounts and the like are sequentially output to the optical signal control unit 422. Note that the third arithmetic circuitry 403b can use a plurality of means as methods of calculating a moving distance and direction. For example, such means include arithmetic circuitry (or software) which measures, as a variation period, a temporal change in light intensity data obtained by each of integrated solid-state image sensors 403a arranged in a matrix, and separates the data into data on the rows (x) and the columns (y), thereby calculating a velocity and a moving distance in each direction. Although it is possible to calculate a shift in a rotating (θ) direction by using a single integrated solid-state imaging device, an arrangement using a plurality of devices is preferable because it increases accuracy.

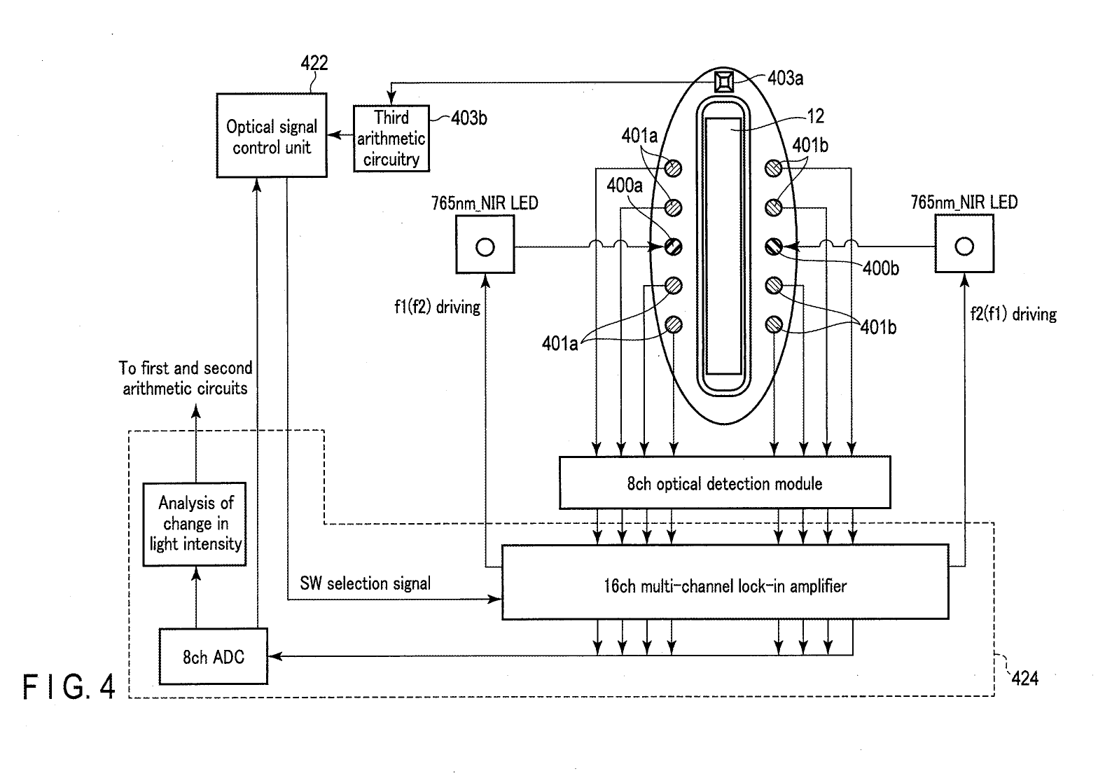

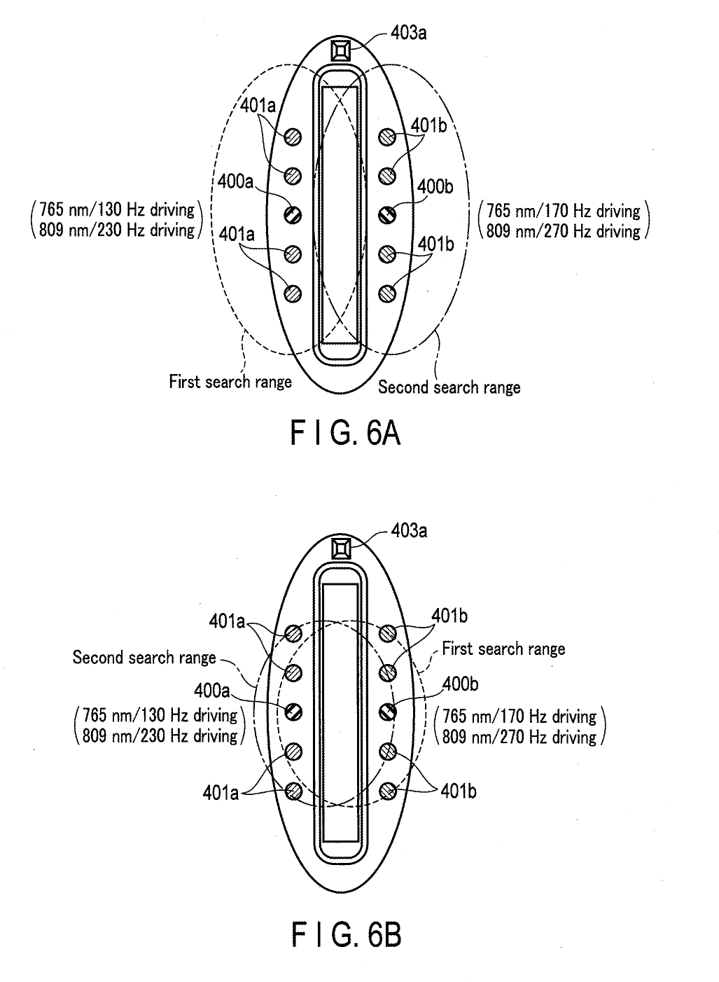

FIG. 4 is a view showing an example of the arrangement of the optical measurement system of the biometric measurement apparatus 4. The optical measurement system of the biometric measurement apparatus 4 will be described in more detail with reference to FIG. 6. As the first light source 420a and the second light source 420b, for example, LEDs with wavelengths near 765 nm are used. The two LEDs are blinked at the different driving frequencies f1 and f2. As signals with the driving frequencies f1 and f2, phase detection reference signals from the multi-channel lock-in amplifier (to be described later) are used. Light beams generated by the first light source 420a and the second light source 420b are transferred from the LEDs to the first light emission unit 400a and the second light emission unit 400b via optical fibers. The first light emission unit 400a and the second light emission unit 400b emit the living body with light. Light from the living body enters each optical detection unit 401 (eight optical detection units in the case shown in FIG. 4) and is transferred to an 8 ch optical detection module constituted by photoelectric conversion elements (a photodiode, avalanche photodiode, phototransistor, and the like) via an optical fiber. Note that using a device having a multiplying effect such as an avalanche photodiode as a photoelectric conversion element can improve the SN ratio. Amplification circuitry converts each of photocurrents with the driving frequencies f1 and f2 into a photocurrent having a proper potential and circuitry impedance. Each photocurrent is then input to a 16 ch multi-channel lock-in amplifier (in the case shown in FIG. 4, a parallel 8 ch optical detection module is connected to a parallel 8×2 (16) ch lock-in amplifier to perform detection with two frequencies). An 8 ch AD converter A/D-converts each photocurrent output from the 16 ch multi-channel lock-in amplifier. The optical analysis unit 424 analyzes a change in the intensity of each photocurrent. These processes are executed for an optical signal acquired at each measurement position along with the movement of the probe P. In parallel with these operations in the optical measurement system, measurement position information based on image data obtained by the integrated solid-state image sensor 403a is sent from the third arithmetic circuitry 403b to the first and second arithmetic circuits 426a and 426b via the optical analysis unit 424. This information is then added as position information to the analysis information described above.

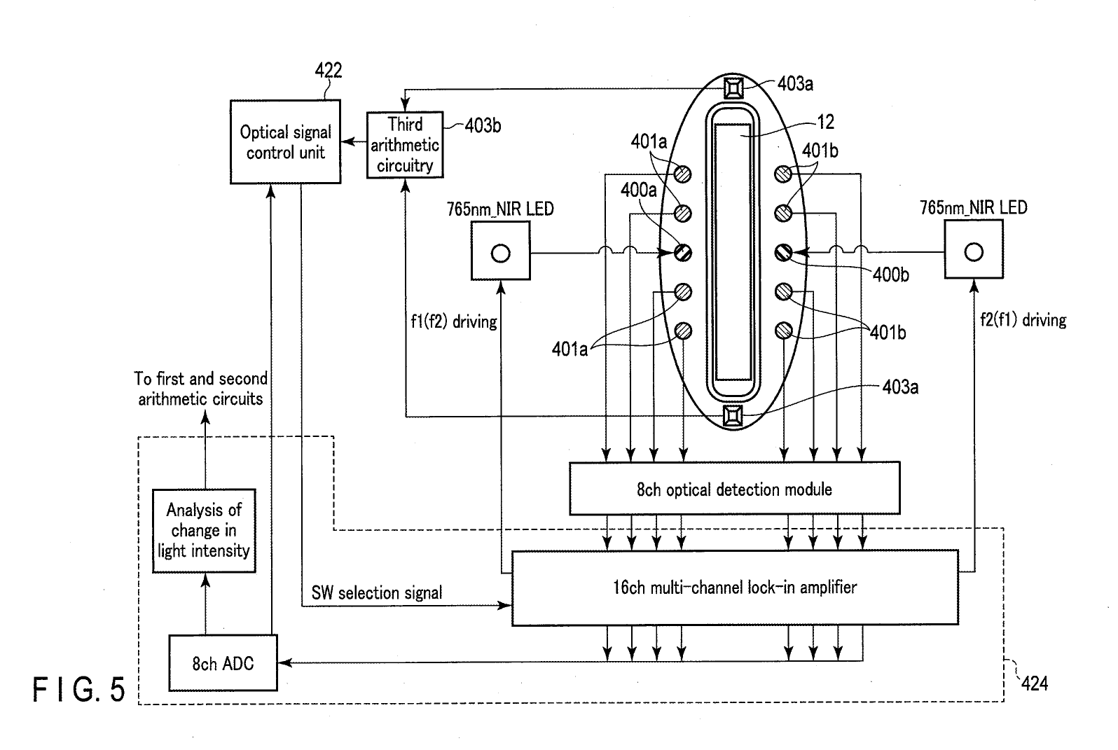

FIG. 5 is a view showing another example of the arrangement of the optical measurement system of the biometric measurement apparatus 4. This example differs from that shown in FIG. 4 in that the integrated solid-state image sensors 403a are arranged at two positions near the respective short sides of the ultrasonic transmission/reception surface, and a third arithmetic circuitry 403b for calculating the moving distances of the integrated solid-state image sensors 403a is provided. It is possible to accurately acquire a change in the rotating direction of the probe P by calculating the moving distances at the two positions and the like by using the integrated solid-state image sensors 403a arranged at the two positions and the third arithmetic circuitry 403b corresponding to each of them.

(Optical Biometric Measurement Accompanied with Movement of Probe P)

The biometric measurement apparatus 4 according to this embodiment executes optical biometric measurement at a plurality of measurement positions (i.e., a series of operations including emitting the inside of the living body with light by the plurality of light emission units 400 and optical detection from the inside of the living body by the plurality of optical detection units 401) while manually moving the probe P. At each measurement position, a moving distance and a moving direction from a reference position (e.g., an initial position or a registered position) are measured by using the integrated solid-state image sensor 403a and the third arithmetic circuitry 403b. This makes it possible to obtain measurement data for each of different positional relationships by changing the positional relationship (or the geometric relationship) between an abnormal region and the light emission unit 400 and the optical detection unit 401 (or ultrasonic transmission/reception surface) for each measurement position. In principle, therefore, it is possible to increase the amount of measurement data.

Note that any technique can be used for optical measurement at each measurement position. This embodiment will exemplify a case in which optical measurement is performed at a plurality of measurement positions in accordance with the rough search mode and the fine adjustment mode, while the probe P is moved, and, for example, the depth of an abnormal region from the surface of the object is calculated by using measurement data at each measurement position. In this case, the rough search mode is a mode for roughly searching for an abnormal region when adjusting the position of the probe P on the surface of the living body so as to make the ultrasonic scan slice of the ultrasonic probe 12 include a diagnostic target region (a region recognized as the abnormal region or suspected to have abnormality) in the living body. The fine adjustment mode is a mode for accurately searching for the abnormal region when adjusting the position of the ultrasonic probe 12 on the surface of the living body.

As shown in FIG. 6A, in the rough search mode, the first light emission unit 400a is paired with a plurality of optical detection units 401a, and the second light emission unit 400b is paired with a plurality of optical detection units 401b (that is, the light emission unit and the detection units arranged on the same side of the ultrasonic probe 12 are paired).

After the pairing, optical biometric measurement is executed at a plurality of measurement positions, while the position of the probe P is changed, by using a region (first search range) searched by the first light emission unit 400a and the plurality of optical detection units 401a and a region (second search range) searched by the second light emission unit 400b and the plurality of optical detection units 401b. At this time, for example, if a signal in a specific frequency band corresponding to a diagnostic target region which is detected from the second search range is higher in intensity than that from the first search range, it indicates that the diagnostic region exists closer to the second search range than the center (long axis) of the ultrasonic probe 12. On the contrary, if such a signal detected from the first search range is higher in intensity than that from the second search range, it indicates that the diagnostic region exists closer to the first search range than the center (long axis) of the ultrasonic probe 12. In the rough search mode, relatively large regions for searching for an abnormal region can be set on the two sides of the ultrasonic emission surface of the ultrasonic probe 12. It can be said from these characteristics that this mode is suitable to guide a diagnostic target region near the ultrasonic probe.

In contrast, the fine adjustment mode is executed after, for example, rough position adjustment is performed through the rough detection mode. As shown in FIG. 6B, in the fine adjustment mode, the first light emission unit 400a is paired with the plurality of optical detection units 401b, and the second light emission unit 400b is paired with the plurality of optical detection units 401a (that is, the light emission units and the detection units arranged on the two sides of the ultrasonic probe 12 are respectively paired).

After the pairing, optical biometric measurement is executed at a plurality of measurement positions, while the position of the probe P is changed, by using a region (first search range) searched by the first light emission unit 400a and the plurality of optical detection units 401b and a region (second search range) searched by the second light emission unit 400b and the plurality of optical detection units 401a. At this time, for example, it is possible to narrow down the position of a diagnostic target region near the center of the probe P by comparing the intensity of a signal in a specific frequency band corresponding to the diagnostic target region which is detected from the first search range with that detected from the second search region. In the fine adjustment mode, a region for searching for an abnormal region is set so as to include the ultrasonic emission surface of the ultrasonic probe 12. It can be said from these characteristics that this mode is suitable to accurately guide a diagnostic target region into the ultrasonic scanning surface.

Note that the rough search mode and the fine adjustment mode are switched from each other by, for example, an operation with the input device 13. However, this is not exhaustive. For example, the apparatus may automatically select the rough search mode or the fine adjustment mode by comparing the distance between a diagnostic target region and the ultrasonic transmission/reception surface with a predetermined threshold.

The optical analysis unit 424 analyzes a change in the intensity of detected light between the optical detection units 401 at the respective measurement positions. The arithmetic circuitry 426 calculates the depth of an abnormal region from the surface of the object which indicates a predetermined light absorption coefficient in the object, the three-dimensional position, and the distance, for each measurement position, based on the analysis result obtained by the optical analysis unit 424 (a change in the intensity of detected light between the optical detection units 401).

When guiding the position of the ultrasonic probe 12 in ultrasonic image diagnosis, an optical signal corresponding to each measurement position in each of the rough search mode and the fine adjustment mode is subjected to predetermined processing in the optical analysis unit 424, first arithmetic circuitry 426a, and second arithmetic circuitry 426b, and the resultant signal is sent as information concerning the position of the diagnostic target region and associated with measurement position information to the scanning support information creation unit 44. Based on the received information concerning the diagnostic target region, the scanning support information creation unit 44 generates and outputs support information for more favorably guiding the position, orientation, posture, pressure, and the like of the probe P with respect to the object and the diagnostic target region. For example, the monitor 14 displays the output support information in a predetermined form, together with the measurement position information.

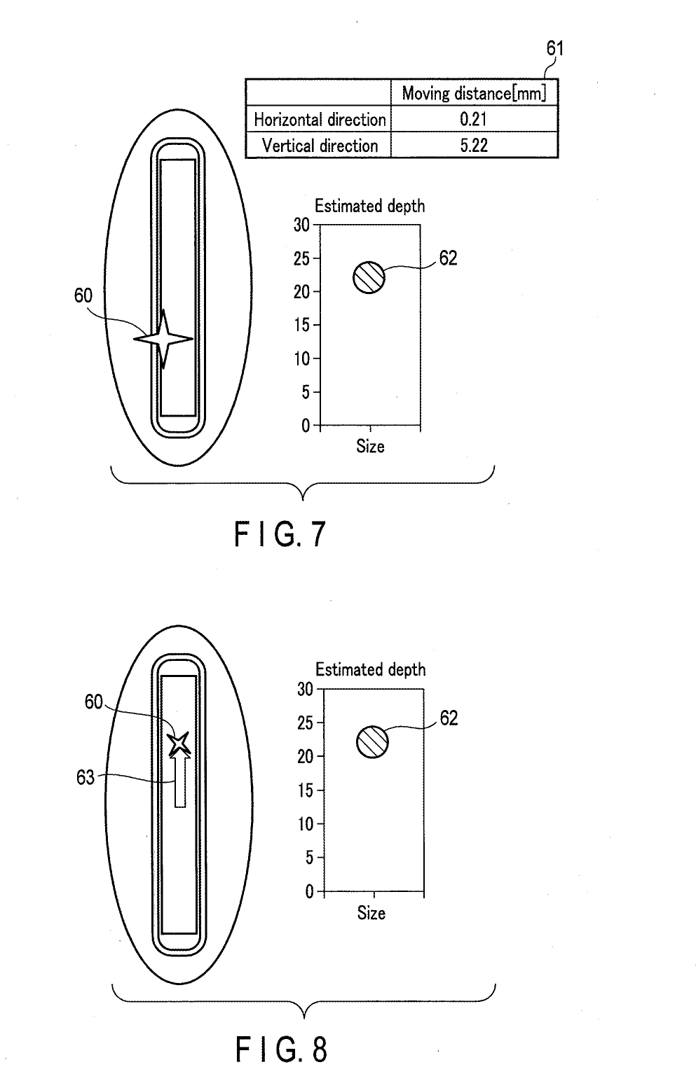

FIG. 7 is a view showing an example of display of optical measurement results and support information for guiding the probe P in the ultrasonic diagnostic apparatus 1 according to this embodiment. In the case shown in FIG. 7, within the object contact surface of the current probe P, as information indicating a specific position immediately below which an (estimated) abnormal region exists, the central position of an (estimated) abnormal region is indicated by a predetermined mark 60 (a cross star in the case shown in FIG. 7) on a schematic view of the contact surface of the probe P with respect to an object. In addition, information concerning the depth of an (estimated) abnormal region from the surface of the object and the size of the region is indicated by a graph and a mark 62 like those shown on the lower right of FIG. 7. Furthermore, the moving distance from the reference position (moving amounts on the surface of the object in the horizontal and vertical directions) is indicated by a correspondence table 61 like that shown on the upper right of FIG. 7.

FIG. 8 is a view showing another example of display of optical measurement results and support information for guiding the probe P in the ultrasonic diagnostic apparatus 1 according to this embodiment. In the case shown in FIG. 8, the direction and length of an arrow 63 indicate navigation information indicating “how much and in which direction the probe P should be moved from the current position to place an (estimated) abnormal region in the center of the ultrasonic scanning slice”, instead of a moving distance from the reference position shown in FIG. 7.

Note that the navigation information indicating “how much and in which direction the probe P should be moved to place an (estimated) abnormal region in the center of the ultrasonic scanning slice” shown in FIG. 8 is not limited to screen information, and may be output as speech information.

### (First Modification)

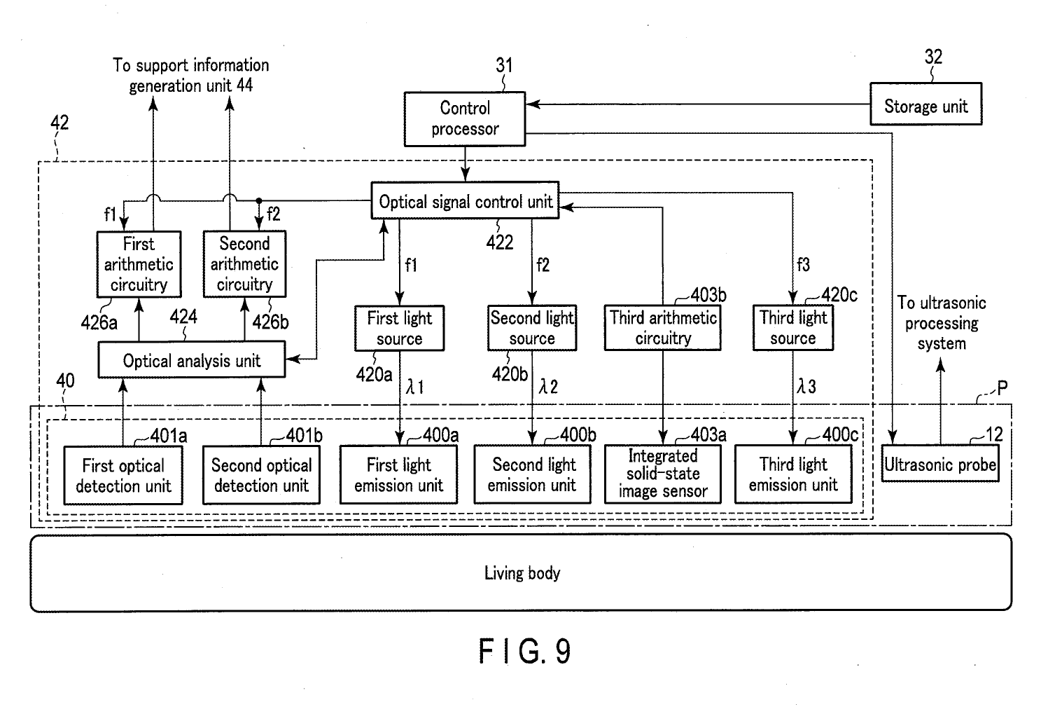

FIG. 9 is a block diagram showing the arrangement of the biometric measurement apparatus 4 according to the first embodiment. As compared with the case shown in FIG. 2, this apparatus further includes, near the integrated solid-state image sensor 403a, a third light emission unit 400c and a third light source 420c as components of an optical system dedicated to imaging performed by the integrated solid-state image sensor 403a. The arrangement using external light (environmental light) shown in FIG. 2 has the merit of being capable of simplifying the arrangement of the probe P, but has the demerit of adding large noise to a signal obtained by the integrated solid-state image sensor integrated solid-state image sensor 403a. If this arrangement includes an optical system (the third light emission unit 400c and the third light source 420c) used exclusively to imaging performed by the integrated solid-state image sensor 403a, like the arrangement shown in FIG. 8, noise can be greatly reduced. When using this arrangement, it is preferable to add an optical filter which transmits light from the exclusive light source to the integrated solid-state image sensor 403a, in order to reduce the influence of external light.

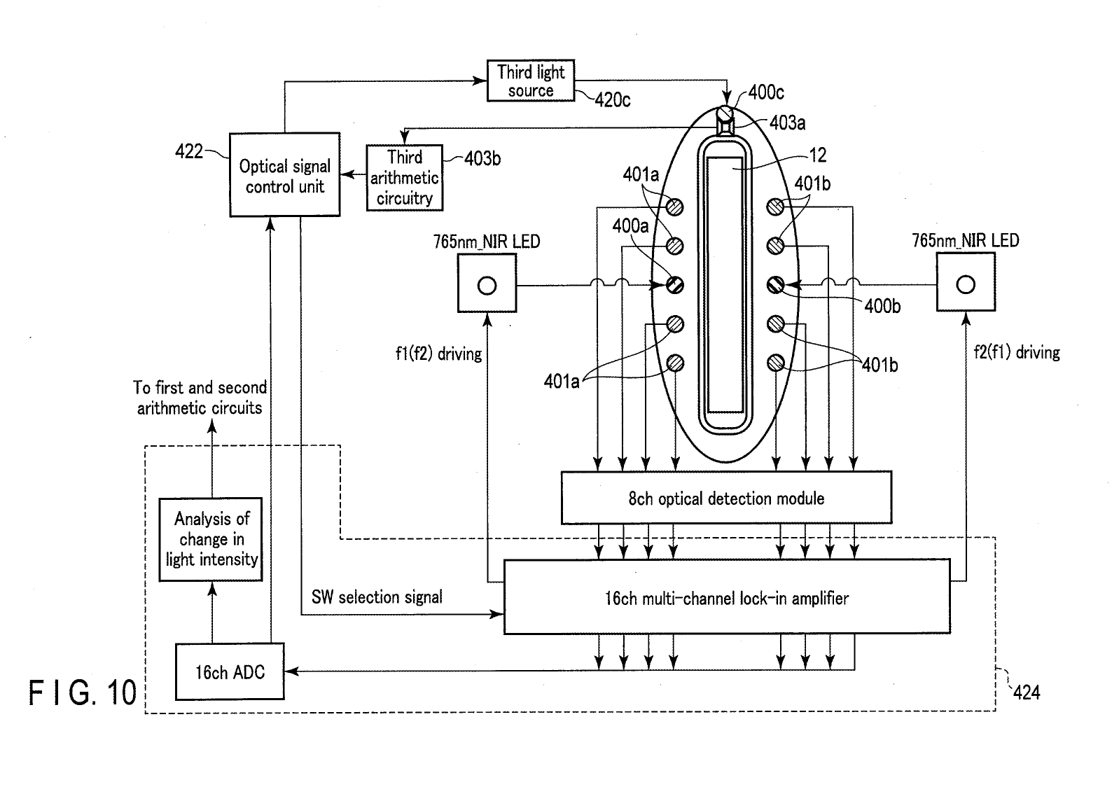

FIG. 10 shows an example of the arrangement of the optical detection system of the biometric measurement apparatus 4 shown in FIG. 9. This example differs from those shown in FIGS. 4 and 5 in that it further includes the third light emission unit 400c and the third light source 420c as components of an optical system dedicated to imaging performed by the integrated solid-state image sensor 403a, and also includes, near the integrated solid-state image sensor 403a, a red (peak at 670 nm) light source and circuitry for controlling the light source. As an optical filter, a bandpass filter which transmits red light (670 nm) or light close in wavelength to red is preferably used.

### (Second Modification)

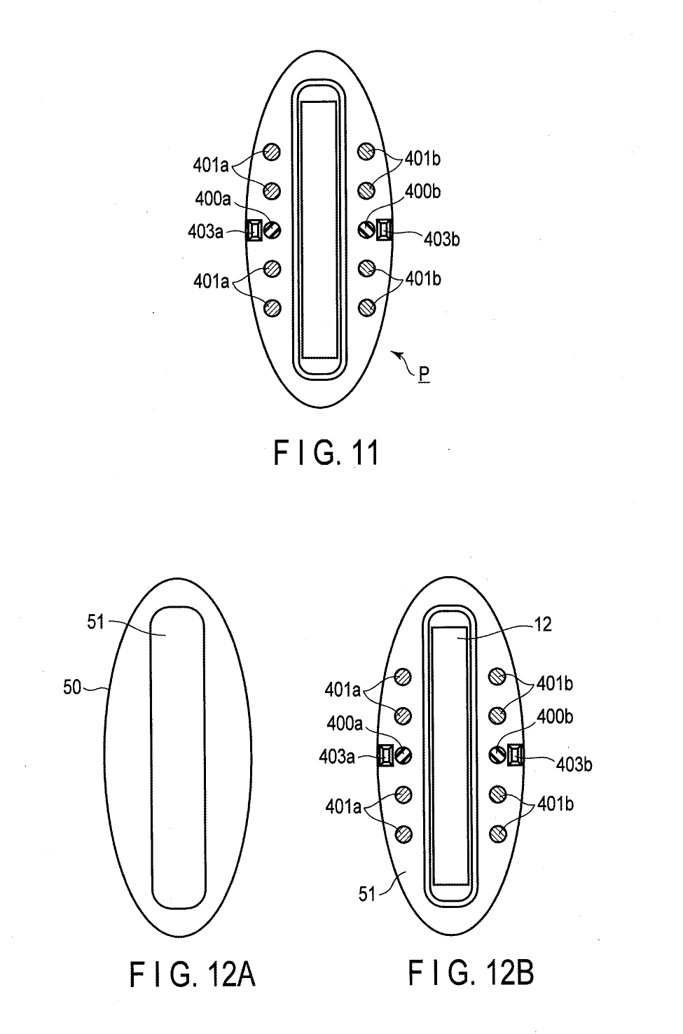

FIG. 11 a view when the probe P according to the second modification is seen from the object contact surface side, showing an example of the placement of the light emission units 400, the optical detection units 401, and the two integrated solid-state image sensors 403a and 403b with respect to the ultrasonic transmission/reception surface of the ultrasonic probe 12. In the case shown in FIG. 11, the integrated solid-state image sensor 403a is arranged on a side of the first light emission unit 400a for biometric component measurement, and the integrated solid-state image sensor 403b is arranged on a side of the second light emission unit 400b for biometric component measurement. Each light emission unit 400 also functions as a light source for imaging performed by each adjacent integrated solid-state image sensor (that is, serving both for biometric component measurement and imaging by each integrated solid-state image sensor). This arrangement needs to be designed to avoid the influence of driving frequencies for biometric component measurement on the integrated solid-state image sensors and the third arithmetic circuitry. More specifically, the first light emission unit 400a and the second light emission unit 400b may be controlled to be blinked during biometric component measurement and always turned on during moving distance measurement using the respective integrated solid-state image sensors. In addition, there is available a method of erasing a driving frequency as noise in the third arithmetic circuitry 403b. Furthermore, there is available a method of separating biometric component measurement light and moving distance measurement light by using an optical filter upon adding reference light to the first light emission unit 400a and the second light emission unit 400b.

### (Third Modification)

FIGS. 12A and 12B are views when the probe P according to the third modification is seen from the object contact surface side. As shown in FIGS. 12A and 12B, the contact surface side of the probe P with respect to an object is provided with an optical measurement sole 50 covering the first light emission unit 400a, the second light emission unit 400b, the first optical detection unit 401a, the second optical detection unit 401b, and the integrated solid-state image sensors 403a and 403b. The optical measurement sole 50 includes a window portion 51 for exposing the ultrasonic transmission/reception surface of the ultrasonic probe 12. The ultrasonic probe 12 is accurately fitted in the window portion. In addition, the ultrasonic transmission/reception surface of the ultrasonic probe 12 and the optical measurement sole are adjusted to the same level. Since as light used for measurement according to this embodiment, light in the near-infrared region is used, the optical measurement sole 50 is formed from a visible light cut filter material. This arrangement has the merit of enabling the optical detection units 401a and 401b and the integrated solid-state image sensors 403a and 403b to detect, as a signal, light whose visible light noise is attenuated by the optical measurement sole 50. In addition, the arrangement can prevent ultrasonic gel from entering the dents of the openings (or light-receiving surfaces) of the integrated solid-state image sensors.

### (Fourth Modification)

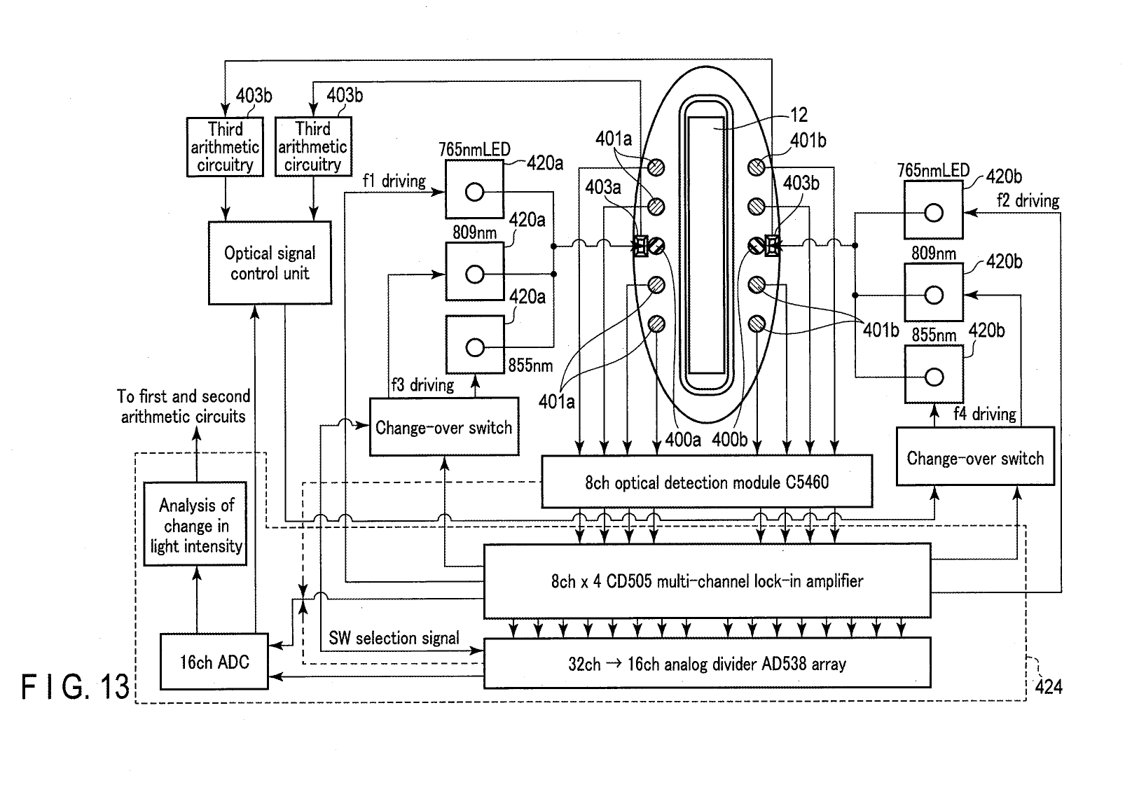

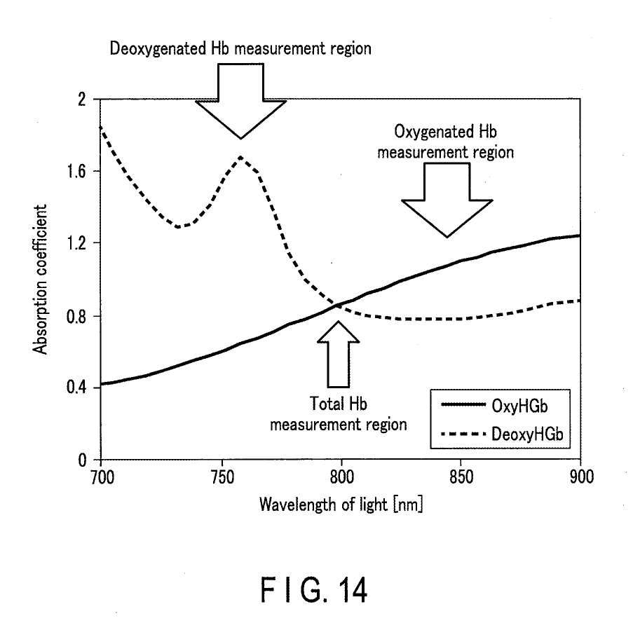

FIG. 13 is a view showing another example of the arrangement of the optical measurement system of the biometric measurement apparatus 4. As shown in FIG. 13, the biometric measurement apparatus 4 according to this modification uses the probe P in FIG. 12 and is configured to guide light beams from three light sources 420a1, 420a2, and 420a3 with wavelengths λ1, λ2, and λ3 to the first light emission unit 400a (λ1=765 nm, λ2=809 nm, and λ3=855 nm in the case shown in FIG. 11) and to guide light beams from three light sources 420b1, 420b2, and 420b3 with wavelengths λ1, λ2, and λ3 to the second light emission unit 400b (λ1=765 nm, λ2=809 nm, and λ3=855 nm in the case shown in FIG. 11). The light source 402a1 is driven at a frequency f1. The light sources 420a2 and 420a3 are driven at a frequency f3. The light source 420b1 is driven at a frequency f2. The light sources 420b2 and 420b3 are driven at a frequency f4. The multi-channel lock-in amplifier supplies driving signals of four frequencies to the respective light sources. This multi-channel lock-in amplifier has four frequencies, and has an arrangement of 8 channels×4. This arrangement is provided with an analog divider which narrows down 32 ch signals into 16 ch signals, and includes two integrated solid-state image sensors for moving distance measurement and two third arithmetic circuits.

Experiments conducted by the present inventors have revealed that when measurement is performed at the same light source position and the same detection position to correct variations in light intensity accompanying deformation caused by pressurization and the like at the time of optical biometric measurement, the correlation relationship between both a wavelength (about 765 nm) for the detection of oxygenated hemoglobin and a wavelength (about 855 nm) for the detection of deoxygenated hemoglobin and a reference wavelength (about 809 nm) in terms of light intensity is nearly a proportional relationship.

Under the circumstances, the biometric measurement apparatus 4 according to this modification adopts a system which divides/approximates the intensity of light having a detection signal wavelength (about 765 nm) by a reference wavelength (about 809 nm) and analyzes the resultant value. At the time of accurate measurement, measurement is performed with a wavelength for the detection of oxygenated hemoglobin (about 765 nm) and a wavelength for the detection of deoxygenated hemoglobin (about 855 nm), and an oxygen saturation is calculated.

According to the arrangement of the ultrasonic diagnostic apparatus or biometric measurement apparatus described above, the following effects can be obtained. That is, a distance from a reference position (e.g., an initial position or registered position) is calculated by using the integrated solid-state image sensors and the third arithmetic circuitry. This makes it possible to obtain measurement data for each of different positional relationships by changing the positional relationship (or the geometric relationship) between an abnormal region and the light emission unit and the optical detection unit (or the ultrasonic transmission/reception surface) for each measurement position. This can increase the amount of measurement data. As a result, it is possible to guarantee the analysis accuracy of measurement information as compared with the related art.

In addition, when performing optical biometric measurement and ultrasonic imaging at a plurality of measurement positions while manually moving the probe P, it is possible to explicitly show the proper position of the probe P to the operator. This can greatly improve the convenience at the time of ultrasonic imaging.

The above described “circuitry” means, for example, a central processing unit (CPU), a graphics processing unit (GPU), an application specific integrated circuit (ASIC), a programmable logical device (e.g., a simple programmable logic device (SPLD), a complex programmable logic device (CPLD), and a field programmable gate array (FPGA)), or the like.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. An ultrasonic diagnostic apparatus comprising:
an ultrasonic probe to transmit an ultrasonic wave from an ultrasonic transmission/reception surface to an object and receive an ultrasonic wave reflected by an inside of the object via the ultrasonic transmission/reception surface;
image generation circuitry to generate an ultrasonic image by using the ultrasonic wave received by the ultrasonic probe;
an optical probe integrally provided with the ultrasonic probe and including at least one light emission circuitry to apply light in an absorption wavelength band of a biological component from a surrounding of the ultrasonic transmission/reception surface onto the inside of the object and a plurality of optical detection circuits to detect an intensity of light having a specific wavelength which is applied from the at least one light emission circuitry and diffused/reflected by the inside of the object;
at least one integrated solid-state image sensor to obtain data with respect to intradermal information of the object; and
analysis circuitry to analyze a change in the light intensity detected by the each optical detection circuit.

2. The apparatus of claim 1, further comprising calculation circuitry to calculate a moving distance and a moving direction of the optical probe based on a temporal change in the data obtained by the at least one integrated solid-state image sensor.

3. The apparatus of claim 2, further comprising a display to display at least one of information concerning a moving distance and a moving direction of the optical probe and information concerning guiding of a position of the ultrasonic probe which is generated based on the moving distance and the moving direction of the optical probe.

4. The apparatus of claim 1, further comprising an optical filter provided on a side of the optical probe which comes into contact with an object surface and transmits light having the specific wavelength.

5. The apparatus of claim 1, wherein the at least one integrated solid-state image sensor obtains information corresponding to the amount of light absorbed which originates from a composition distribution inside the object.

6. The apparatus of claim 1, wherein the at least one first light emission circuitry applies composite light including light beams whose intensity peaks differ in wavelength position in spectra.

7. The apparatus of claim 1, further comprising at least second light emission circuitry provided near the at least one integrated solid-state image sensor.

8. A biometric examination apparatus comprising:
an optical probe including at least one light emission circuitry to apply light in an absorption wavelength band of a biological component onto an inside of an object and a plurality of optical detection circuits to detect an intensity of light having a specific wavelength which is applied from the at least one light emission circuitry and diffused/reflected by the inside of the object;
at least one integrated solid-state image sensor to obtain data with respect to intradermal information of the object; and
analysis circuitry to analyze a change in the light intensity detected by the each optical detection circuit.
